(19) United States
(12) Patent Application Publication
(10) Pub. No.: US 2013/0266628 A1
(43) Pub. Date: Oct. 10, 2013

(54) GRAPHENE-BASED ANTIMICROBIAL COMPOSITES

(71) Applicants: Pradeep THALAPPIL, Chennai (IN); Theruvakkattil SREENIVASAN SREEPRASAD, Kottayam (IN); Mundampra MALIYEKKAL SHIHABUDHEEN, Chennai (IN)

(72) Inventors: Pradeep THALAPPIL, Chennai (IN); Theruvakkattil SREENIVASAN SREEPRASAD, Kottayam (IN); Mundampra MALIYEKKAL SHIHABUDHEEN, Chennai (IN)

(73) Assignee: INDIAN INSTITUTE OF TECHNOLOGY MADRAS, Chennai (IN)

(21) Appl. No.: 13/443,408

(22) Filed: Apr. 10, 2012

(51) Int. Cl. A01N 59/00; A01N 25/08; C12N 5/09; C12Q 1/02; G01N 21/64; A01N 37/18; A01P 1/00; B82Y 15/00

(52) U.S. Cl. 424/409; 424/125; 514/2.3; 514/2.5; 435/375; 435/29; 435/369; 435/366; 435/372; 977/838; 977/927

(57) ABSTRACT

Composite materials comprising functionalized graphenes, methods of preparing the composite materials, and methods of using the composite materials are described herein. A composite material comprising reduced graphene oxide, chitosan, and native lactoferrin show much higher antimicrobial activity versus individual reduced graphene oxide, chitosan, and native lactoferrin alone.

### BACKGROUND

Nanomaterials such as silver nanoparticles, titanium dioxide nanoparticles, and carbon nanotubes are known antibacterial agents. While each of these shows appreciable antibacterial efficiency, their possible applications are limited by cytotoxicity. Graphene and its chemical analogues, graphene oxide and reduced graphene oxide, may have considerably less cytotoxicity, bringing forth more viable alternatives.

### SUMMARY

Some embodiments describe a composite material comprising: at least one functionalized graphene material selected from graphene, graphene oxide, reduced graphene oxide, and any combination thereof; and at least one transferrin protein selected from native lactoferrin, hololactoferrin, apolactoferrin, serum transferrin, and any combination thereof.

Some embodiments describe a formed article comprising: a composite material having at least one functionalized graphene material selected from graphene, graphene oxide, reduced graphene oxide, and any combination thereof; and at least one protein selected from native lactoferrin, hololactoferrin, apolactoferrin, serum transferrin, and any combination thereof.

Further embodiments describe a method of preparing a composite material comprising: providing at least one functionalized graphene material selected from graphene oxide, reduced graphene oxide, sulfonated graphene, sulfonated graphene oxide, sulfonated reduced graphene oxide, and any combination thereof; providing at least one protein selected from native lactoferrin, hololactoferrin, apolactoferrin, serum transferrin, and any combination thereof; and contacting the at least one functionalized graphene material with the at least one protein to produce the composite material.

Other embodiments describe a method of targeting a cell with an imaging or therapeutic agent, wherein the cell is contacted with the composite material described in other embodiments.

In an embodiment, a method of treating an area containing or thought to contain microorganisms comprises: providing a composite material comprising at least one functionalized graphene and at least one protein or peptide; and contacting an area containing or thought to contain microorganisms with the composite material, whereby microorganisms in the contacted area are exterminated or their growth rate is inhibited.

### DETAILED DESCRIPTION

Methods described herein include novel and simple processes comprising, among other things, the production of composite materials based on functionalized graphene materials.

In embodiments, a functionalized graphene material may be selected from graphene, graphene oxide, reduced graphene oxide, sulfonated graphene, sulfonated reduced graphene oxide, and any combination thereof.

In an embodiment, a composite material may comprise at least one functionalized graphene material and at least one protein or peptide.

In some embodiments, a protein may be selected from natural resistance-associated macrophage protein (NRAMP), rat beta defensins 1 and 2 (RBD-1 and RBD-2), Bin1b, a cathelicidin (rCRAMP), and the defensin-like molecule E-3. In other embodiments, a protein may be a transferrin protein. In these embodiments, a transferrin protein may be selected from native lactoferrin, hololactoferrin, apolactoferrin, serum transferrin, and any combination thereof.

In embodiments, a peptide may be selected from common antimicrobial peptides such as maximin H5, dermcidin, cecropins, andropin, moricin, ceratotoxin, melittin, magainin, dermaseptin, bombinin, brevinin-1, esculentins and buforin II, CAP18, LL37, abaecin, apidaecins, prophenin, indolicidin, brevinins, protegrin, tachyplesins, defensins, and drosomycin. In embodiments, the source of the peptide may be insects, amphibians, reptiles, frogs, honeybees, pigs, cattle, horseshoe crabs, humans, fruit flies seaweeds, shrimp, millipedes, spiders, lobsters, or crayfish.

In some embodiments, the at least one functionalized graphene material may be graphene oxide and the at least one protein may be native lactoferrin. In some embodiments, the at least one functionalized graphene material may be reduced graphene oxide and the at least one protein may be native lactoferrin. In some embodiments, the composite material may be antimicrobial. In some embodiments, the composite material may be present as a free-standing film, a coating on a substrate, an embedded constituent of a second composite material, a nanoparticle, a microparticle, or any combination thereof.

In some embodiments, a composite material comprising reduced graphene oxide, native lactoferrin and chitosan is described. The methods described herein may allow facile production of antimicrobial packaging materials and targeted medical devices with applications in imaging and therapy.

In some embodiments, the composite material may further comprise at least one polymer selected from biopolymers and conducting polymers. Exemplary conducting polymers include polyacetylene, polyphenylene vinylene, polypyrrole, polythiophene, polyaniline, polyphenylene sulfide, and their derivatives. In embodiments, the combination of graphene and a conducting polymer may provide a conductive composite material, wherein the conductivity may be enhanced relative to graphene or the conductive polymer alone. In some embodiments, the composite material may further comprise chitosan. In embodiments, the addition of chitosan to the composite material may provide an antimicrobial material, wherein the antimicrobial activity may be enhanced relative to chitosan alone or the composite material without chitosan.

In some embodiments, the composite material may further comprise at least one metal cluster. In embodiments the addition of a metal cluster to the composite material may provide photoluminescence to the composite material and may enhance the antimicrobial activity of the composite material relative to the metal cluster alone or the composite material without the metal cluster. In some embodiments, the at least one metal cluster comprises gold.

In some embodiments, the composite material further comprises at least one fluorophore, magnetic nanomaterial, gadolinium compound, or pharmaceutical agent. The addition a fluorophore, a magnetic nanomaterial, or a gadolinium compound to the composite material may provide an additional means of imaging the composite material. Exemplary imaging methods include fluorescence-based methods, microscopy, x-ray, computed tomography (CT), and magnetic resonance imaging (MRI). The addition of a pharmaceutical agent to the composite material may allow the composite material to be used as a therapeutic drug delivery device. In some embodiments, at least one pharmaceutical agent and at least one fluorophore, magnetic nanomaterial, or gadolinium compound may be added to the composite material. The pharamaceutical agent can generally be any drug or prodrug.

In an embodiment, a formed article may comprise a composite material having at least one functionalized graphene material and at least one protein or peptide. In embodiments, the composite material may further comprise any of the materials or functionalities described in the preceding embodiments or combinations thereof. In some embodiments, the formed article may be a food packaging material, a filter medium, or a targeted medical device.

In an embodiment, a method of preparing a composite material comprises: providing at least one functionalized graphene material; providing at least one protein; and contacting the at least one functionalized graphene material with the at least one protein to produce the composite material.

In some embodiments, the at least one functionalized graphene material may be dispersed in an aqueous solution with a pH of about 4.5 to about 8.5, about 5 to about 7, or about 6.0 to about 6.5. In some embodiments, the at least one functionalized graphene material may be dispersed in an aqueous solution with a pH of about 4.5, about 5, about 5.5, about 6, about 6.5, about 7, about 7.5, about 8, about 8.5, or any pH value or range of values between those listed (inclusive of endpoints).

In some embodiments, the at least one functionalized graphene material may be present in a dispersion at about 0.01 weight percent to about 0.1 weight percent. In some embodiments, the at least one functionalized graphene material may be present in a dispersion at a weight percent of about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, or any percentage or range of percentages between those listed (inclusive of endpoints).

In some embodiments, the at least one functionalized graphene material may be contacted with the at least one protein for about 1 hours, about 1.5 hours, about 2 hours about 2.5 hours about 3 hours about 3.5 hours, about 4 hours, about 4.5 hours, about 5 hours, or any time or range of times between those listed (inclusive of endpoints).

In some embodiments, the at least one functionalized graphene material may be sulfonated reduced graphene oxide, and the method may further comprise contacting reduced graphene oxide with a 4-sulfobenzenediazonium salt prior to contacting with the functionalized graphene material with the at least one protein. In these embodiments, the reduced graphene oxide may be present in a dispersion at about 0.01 weight percent to about 0.1 weight percent. In some embodiments, the reduced graphene oxide may be present in a dispersion at a weight percent of about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, or any percentage or range of percentages between those listed (inclusive of endpoints). In some embodiments, the reduced graphene oxide may be contacted with the 4-sulfobenzenediazonium salt for about 1 hour, about 1.5 hours, about 2 hours about 2.5 hours about 3 hours about 3.5 hours, about 4 hours, about 4.5 hours, about 5 hours, or any time or range of times between those listed (inclusive of endpoints). In some embodiments, the reduced graphene oxide may be contacted with the 4-sulfobenzenediazonium salt at about 0° C., about 2° C., about 4° C., about 6° C., about 8° C., about 10° C., or any temperature or range of temperatures between those listed (inclusive of endpoints). In some embodiments, the 4-sulfobenzenediazonium salt may be produced by contacting sulfanilic acid with sodium nitrite.

In some embodiments, the method may further comprise contacting the composite material with chitosan. In some embodiments, the chitosan may be in a solution comprising acetic acid. In some embodiments, the chitosan may be present in solution at a weight percent of about 0.2 wt. %, about 0.4 wt. %, about 0.6 wt. %, about 0.8 wt. %, about 0.9 wt. %, about 1 wt. %, about 1.2 wt. %, or any percentage or range of percentages between those listed (inclusive of endpoints). In some embodiments, the solution may comprise acetic acid at a mole percent of about 1, about 1.25, about 1.5, about 1.75, about 2, about 2.25, or any mole percent or range of mole percentages between those listed (inclusive of endpoints). In some embodiments, the composite material may be present at about 0.02 weight percent in a first solution; the chitosan may be present at about 0.8 weight percent in a second solution comprising about 1.5 molar acetic acid; and the first solution and second solution may be combined at a volumetric ratio of about 1 to about 0.1, about 1 to about 0.3, about 1 to about 0.5, about 1 to about 0.7, or any ratio or range of ratios between those listed (inclusive of endpoints). In some embodiments, contacting the composite material with chitosan occurs with mixing. In some embodiments, the composite material may be contacted with chitosan for about 1 hours, about 1.5 hours, about 2 hours about 2.5 hours about 3 hours about 3.5 hours, about 4 hours, about 4.5 hours, about 5 hours, or any time or range of times between those listed (inclusive of endpoints).

In an embodiment, a method of targeting a cell with an imaging or therapeutic agent comprises: providing a composite material wherein the composite material comprises, at least one functionalized graphene material, at least one transferrin protein, and at least one fluorophore, magnetic nanomaterial, gadolinium compound, or pharmaceutical agent; and contacting the cell with the composite material, whereby the cell may be targeted with an imaging or therapeutic agent. In some embodiments, the cell may overexpress transferrin receptors. In these embodiments, a cell that overexpresses transferrin receptors may be selectively targeted by the composite material relative to other cells and the imaging or therapeutic agent may be selectively delivered to the overexpressing cells. In some embodiments, the cell may be of a cell line selected from MDA-MB-435, MDA-MB-468, LXFL 592, L292, K562, HeLa, H-Meso, HL60, Hep2, KB-3-1, KB-8-5, KB-C1, KB-V1,MCF-7, scU87Mg, U251MG, K562, MOLT4, or combinations thereof. In some embodiments, the cell may be a cancerous cell. In these embodiments the cancerous cell may comprise a brain tumor.

In an embodiment, a method of treating an area containing or thought to contain microorganisms comprises: providing a composite material comprising at least one functionalized graphene and at least one protein or peptide; and contacting an area containing or thought to contain microorganisms with the composite material, whereby microorganisms in the contacted area are exterminated or their growth rate is inhibited.

### EXAMPLES

### Example 1

### Pre-Oxidation of Graphite

In a 250 mL beaker, concentrated H₂SO₄ (25 mL), 5 g of K₂S₂O₈ and 5 g of P₂O₅ were heated to 90° C. with constant stirring. After the reactants were completely dissolved, the reaction temperature was decreased to 80° C. To this reaction mixture, 6 g of graphite powder was added slowly. Bubbling was observed initially and subsided subsequently, over a period of 30 minutes. The temperature of the reaction mixture was maintained at 80° C. for 5 hours. Heating was stopped and the mixture was diluted with 1 L of distilled water and left undisturbed for 12 hours. The resultant solution was then filtered and washed to remove excess acid. The solid pre-oxidized graphite was dried in air for 12 hours.

### Example 2

### Oxidation to Graphene Oxide

230 mL of concentrated H₂SO₄ was maintained at 0° C. using an ice bath. Pre-oxidized graphite from Example 1 was then added and stirred. 15 g of KMnO₄ was added slowly making sure that the temperature never went above 10° C. The temperature was then raised to 35° C. and allowed to react for 2 hours. Subsequently, 1 L of distilled water was added, carefully keeping the temperature below 50° C. The reaction mixture was again stirred for 2 more hours and then 1.5 L of distilled water and 25 mL of 30% H₂O₂ were added. The mixture was kept at room temperature for 24 hours and the supernatant was decanted. The remaining suspension was centrifuged and washed with 10% HCl followed by distilled water. This was repeated several times. The resultant solid was dried and a 2% (w/w) dispersion was prepared in distilled water. This dispersion was purified by dialysis for 3 weeks to remove all unwanted contaminants like salts and acid. Next, the dispersion of graphene oxide was diluted to 0.1% (w/w) with distilled water.

### Example 3

### Hydrothermal Deoxygenation of Graphene Oxide to Reduced Graphene Oxide Nanosheets

Graphene oxide from Example 2 was sonicated for 45 minutes (CREST TRU-SWEEP 27D, 50 Hz) to exfoliate the suspension completely and centrifuged at 5,000 rpm to remove any unexfoliated graphene oxide. About 50 mL of the purified exfoliated graphene oxide solution (0.05 wt %) was transferred to a Teflon-lined hydrothermal reaction vessel and heated at 180° C. for 6 hours. After 6 hours, the vessel was cooled to room temperature. A black precipitate of reduced graphene oxide settled at the bottom. The purified reduced graphene oxide sheets were redispersed in distilled water by mild sonication.

### Example 4

### Sulfonation of Reduced Graphene Oxide

To increase the stability of graphene in water, sulfonic acid groups were introduced onto reduced graphene oxide surface through a simple sulfonation procedure. 20 mg of sulfanilic acid and 8 mg sodium nitrite were dissolved in a NaOH solution (0.25%). Next, 4 mL of 0.1 M HCl was added to the above mixture and kept in an ice bath under stirring to prepare a aryl diazonium salt solution. After 15 minutes, the aryl diazonium salt solution was added to 20 mL, of a 0.5 mg/mL dispersion of reduced graphene oxide from Example 3 with continuous stiffing for 2 hours in an ice bath. After 2 hours, the solution was filtered and washed and redispersed in distilled water with a final concentration of 0.05 wt. %. The pH of the solution was measured to be around 6.

### Example 5

### Preparation of Reduced Graphene Oxide-Native Lactoferrin Composites

Native lactoferrin from bovine milk was anchored onto reduced graphene oxide substrates through a simple electrostatic interaction. To 5 mL graphene oxide (0.02 wt %) from Example 3, 50, 100, 175 and 250 μL of a native lactoferrin solution (12 mg/mL) were added. The mixtures containing reduced graphene oxide-native lactoferrin composites were stirred for 2 hours and stored at 4° C. for further use.

### Example 6

### Preparation of Reduced Graphene Oxide-Native Lactoferrin-Chitosan Composite

Reduced graphene oxide-native lactoferrin composites from Example 5 were mixed with a chitosan solution (0.8% chitosan in 1.5% acetic acid) in a 1:0.3 ratios (v/v). The mixture was stirred continuously for 2 hours. After 2 hours, the homogeneous dispersions of reduced graphene oxide-native lactoferrin-chitosan composites were kept at 4° C. for further use.

### Example 7

### Preparation of Reduced Graphene Oxide-Chitosan-Native Lactoferrin Films

The solution from Example 5 was transferred to a Petri dish and kept in an oven maintained at 40° C. The mixture was allowed to dry and after complete drying, the film was immersed in ammonia solution (5 vol %) for 15 minutes. Then, the films were washed repeatedly with distilled water to remove ammonia. This film was peeled off for further use.

### Example 8

### Kerley-Born Diffusion Test

In order to qualitatively evaluate the antibacterial ability of the composite, a Kerley-Born diffusion test was carried out. A bacterial (E. coli) test dilution was seeded into Petri dish with EMB agar and spread all over the agars with the help of L-shaped glass rod. Then two paper disks impregnated with a 300 μL of reduced graphene oxide-chitosan-native lactoferrin and graphene oxide-chitosan-native lactoferrin were placed on the agar medium. The impregnated sample diffused around the paper disk, forming a radial decreasing concentration gradient of the sample. The Petri dishes were incubated for 16 hours at 37° C. and the ability of the sample to inhibit the growth of the organism was indicated by a zone of inhibition around the disk. The reduced graphene oxide-chitosan-native lactoferrin and graphene oxide-chitosan-native lactoferrin composite materials showed almost wiped out the E. coli around the disk. This was an improvement compared to the individual reduced graphene oxide and graphene oxide materials alone, which showed some inhibition of growth versus a control.

This disclosure is not limited to the particular systems, devices and methods described, as these may vary. The terminology used in the description is for the purpose of describing the particular versions or embodiments only, and is not intended to limit the scope. While various compositions and methods are described in terms of “comprising” various components or steps (interpreted as meaning “including, but not limited to”), the compositions and methods can also “consist essentially of” or “consist of” the various components and steps, and such terminology should be interpreted as defining essentially closed-member groups.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, or claims, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 substituents refers to groups having 1, 2, or 3 substituents. Similarly, a group having 1-5 substituents refers to groups having 1, 2, 3, 4, or 5 substituents, and so forth.

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

What is claimed is:

1. A composite material comprising:
at least one functionalized graphene material selected from graphene, graphene oxide, reduced graphene oxide, and any combination thereof; and
at least one protein or peptide.

2. The composite material of claim 1, wherein the at least one protein or peptide is a transferrin protein selected from native lactoferrin, hololactoferrin, apolactoferrin, serum transferrin, and any combination thereof.

3. The composite material of claim 1, further comprising at least one polymer selected from biopolymers and conducting polymers.

4. The composite material of claim 1, further comprising chitosan.

5. The composite material of claim 1, wherein the at least one functionalized graphene material is graphene oxide and the at least one protein is native lactoferrin.

6. The composite material of claim 1, wherein the at least one functionalized graphene material is reduced graphene oxide and the at least one protein is native lactoferrin.

7. The composite material of claim 1, wherein the composite material is antimicrobial.

8. The composite material of claim 1, wherein the composite material further comprises at least one fluorophore, magnetic nanomaterial, gadolinium compound, or pharmaceutical agent.

9. The composite material of claim 1, wherein the composite material is present as a free-standing film, a coating on a substrate, an embedded constituent of a second composite material, a nanoparticle, a mircoparticle, or any combination thereof.

10. A formed article comprising a composite material comprising:
at least one functionalized graphene material selected from graphene, graphene oxide, reduced graphene oxide, and any combination thereof; and
at least one protein selected from native lactoferrin, hololactoferrin, apolactoferrin, serum transferrin, and any combination thereof.

11. The formed article of claim 10, wherein the composite material further comprises a polymer selected from biopolymers and conducting polymers.

12. The formed article of claim 10, wherein the composite material further comprises chitosan.

13. The composite material of claim 10, wherein the composite material further comprises at least one fluorophore, magnetic nanomaterial, gadolinium compound, or pharmaceutical agent.

14. The formed article of claim 10, wherein the formed article is a food packaging material, a filter medium, or a targeted medical device.

15. A method of preparing a composite material, the method comprising:
providing at least one functionalized graphene material selected from graphene oxide, reduced graphene oxide, sulfonated graphene, sulfonated graphene oxide, sulfonated reduced graphene oxide, and any combination thereof;
providing at least one protein selected from native lactoferrin, hololactoferrin, apolactoferrin, serum transferrin, and any combination thereof; and
contacting the at least one functionalized graphene material with the at least one protein to produce the composite material.

16. The method of claim 15, wherein the at least one functionalized graphene material is sulfonated reduced graphene oxide, and the method further comprises contacting reduced graphene oxide with a 4-sulfobenzenediazonium salt prior to contacting with the at least one protein.

17. The method of claim 15, further comprising contacting the composite material with chitosan.

18. A method of targeting a cell with an imaging or therapeutic agent, the method comprising:
providing a composite material wherein the composite material comprises,
at least one functionalized graphene material selected from graphene, graphene oxide, reduced graphene oxide, and any combination thereof,
at least one transferrin protein selected from native lactoferrin, hololactoferrin, apolactoferrin, serum transferrin, and any combination thereof, and
at least one fluorophore, magnetic nanomaterial, gadolinium compound, or pharmaceutical agent; and
contacting the cell with the composite material, whereby the cell is targeted with an imaging or therapeutic agent.

19. The method of claim 18, wherein the cell overexpresses transferrin receptors.

20. The method of claim 18, wherein the cell is of a cell line selected from MDA-MB-435, MDA-MB-468, LXFL 592, L292, K562, HeLa, H-Meso, HL60, Hep2, KB-3-1, KB-8-5, KB-C1, KB-V1, MCF-7, scU87Mg, U251MG, K562, MOLT4, or combinations thereof.

21. The method of claim 18, wherein the cell is a cancerous cell.

22. A method of treating an area containing or thought to contain microorganisms, the method comprising:
providing a composite material wherein the composite material comprises,
at least one functionalized graphene material selected from graphene, graphene oxide, reduced graphene oxide, and any combination thereof,
at least one protein or peptide; and
contacting an area containing or thought to contain microorganisms with the composite material, whereby microorganisms in the contacted area are exterminated or their growth is inhibited.
